(19) United States
(12) Patent Application Publication
(10) Pub. No.: US 2024/0065807 A1
(43) Pub. Date: Feb. 29, 2024

(54) 3D FACIAL RECONSTRUCTION AND VISUALIZATION IN DENTAL TREATMENT PLANNING

(71) Applicant: Align Technology, Inc., San Jose, CA (US)

(72) Inventors: Michael Chang, Zürich (CH); Niko Benjamin Huber, Zug (CH); Eric Paul Meyer, Zürich (CH); Petri Tanskanen, Schlieren (CH); Olivier Saurer, Zürich (CH)

(21) Appl. No.: 18/239,712

(22) Filed: Aug. 29, 2023

Related U.S. Application Data
(60) Provisional application No. 63/373,921, filed on Aug. 30, 2022.

(51) Int. Cl. A61C 7/00; A61B 1/00; A61B 1/24; G06T 17/00; G06T 19/00

(52) U.S. Cl. CPC A61C 7/002; A61B 1/000096; A61B 1/24; G06T 17/00; G06T 19/00; G06T 2210/41

(57) ABSTRACT

The present disclosure is directed to capturing a 3-dimensional representation of a patient's face from multiple angles to be integrated with (fused) with at least a 3-dimensional intra-oral mesh of the patient's teeth for purposes of visualizing a dental treatment plan. In one aspect, a method includes capturing media of a patient's face from multiple angles, using at least one device; transforming the media into a 3-dimensional representation of the patient's face; and transmitting, the 3-dimensional representation of the patient's face to one or more processing components to be integrated with a 3-dimensional representation of the patient's intra-oral scan to visualize a dental treatment plan for the patient.

### RELATED APPLICATIONS

The present application claims the benefit under 35 U.S.C. § 119(e) of U.S. Provisional Patent Application No. 63/373,921, filed Aug. 30, 2022, which is incorporated by reference herein.

### TECHNICAL FIELD

The subject matter of this disclosure relates in general to the field of dental treatment planning, and more specifically to capturing a 3-dimensional representation of a patient's face across multiple angles using a mobile device to provide a complete visual display of a dental treatment plan to be implemented.

### BACKGROUND OF THE INVENTION

Dental treatment procedures typically involve repositioning a patient's teeth to a desired arrangement in order to devise and implement a treatment plan. To achieve these objectives, various in-office imaging tools and devices are utilized. Existing tools and systems may provide a 3-dimensional representation of arrangement of teeth, which a doctor may utilize to determine how a treatment may impact teeth positioning and shapes. Existing tools may not take into account facial relationships between the positions and orientations of teeth and the shape and position of facial features of a patient.

Current treatment planning processes may take into account facial relationships between the 2-dimensional positions and orientations of teeth and the shape and position of facial features of a patient.

### SUMMARY OF THE INVENTION

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides tools for capturing a 3-dimensional representation of a patient's face across multiple angles (e.g., using a mobile device). The 3-dimensional representation may then be integrated with (fused) with at least a 3-dimensional intra-oral mesh of the patient's teeth. The resulting high resolution combined representation may then be integrated with one or more 3-dimensional treatment planning products, that can provide accurate and realistic results of a dental treatment plan for the patient.

In one aspect, a method includes capturing media of a patient's face from multiple angles, using at least one device; transforming the media into a 3-dimensional representation of the patient's face; and transmitting, the 3-dimensional representation of the patient's face to one or more cloud-based processing components to be integrated with a 3-dimensional representation of the patient's intra-oral scan to visualize a dental treatment plan for the patient.

In another aspect, the at least one device is a mobile device with a built-in camera.

In another aspect, the media includes a plurality of 2-dimensional images of the patient's faced captured by moving the mobile device around the patient's face.

In another aspect, the media includes a video of the patient's face.

In another aspect, the video is captured using a multi-camera system configured to capture images of the patient's face from a plurality of angles at the same time, the at least one device being the multi-camera system.

In another aspect, the 3-dimensional intra-oral scan is used as a reference for scaling the 3-dimensional representation of the patient's face.

In another aspect, the media is captured via an application installed on the at least one device having access to a built-in camera of the at least one device.

In another aspect, the application provides real-time guidance for moving the at least one device around the patient's face in order to optimize the media captured.

In another aspect, the media includes a plurality of 2-dimensional images of the patient's face, and transforming the media into the 3-dimensional representation of the patient's face comprises: determining a plurality of camera poses, where each camera pose of the plurality of camera poses is determined for a 2-dimensional image of the plurality of 2-dimensional images; generating a plurality of depth maps, where each depth map of the plurality of depth maps is generated for a 2-dimensional image of the plurality of 2-dimensional images based at least in part on the camera pose for the 2-dimensional image; and generating the 3-dimensional representation of the patient's face based on combining information from the plurality of depth maps.

In one aspect, a method of generating a 3-dimensional representation of a patient's face for dental treatment planning includes receiving, at a processing component communicatively coupled to at least one device, a 3-dimensional representation of the patient's face based on media of the patient's face captured from multiple angles by the at least one device; and integrating, at the processing component, the 3-dimensional representation of the patient's face with a 3-dimensional representation of the patient's intra-oral scan to visualize a dental treatment plan for the patient.

In another aspect, integrating the 3-dimensional representation of the patient's face with the 3-dimensional representation of the patient's intra-oral scan, includes identifying inner mouth region of the patient in the 3-dimensional representation of the patient's face; removing the inner mouth region from the 3-dimensional representation of the patient's face; determining a scaled-rigid relative transform for the 3-dimensional representation of the patient's face to the 3-dimensional representation of the patient's intra-oral scan; replacing the inner mouth region removed from the 3-dimensional representation with the 3-dimensional representation of the intra-oral scan; and aligning the 3-dimensional representation of the patient's face with the 3-dimensional representation of the patient's intra-oral scan based on the scaled-rigid relative transform.

In another aspect, the inner mouth region is identified in a 2-dimensional space using a machine learning-based model.

In another aspect, the 3-dimensional intra-oral scan provides a visualization of the patient's teeth after the dental treatment plan is completed.

In one aspect, a system includes at least one device; and a processing component communicatively coupled to the at least one device. The at least one device is configured to capture media of a patient's face from multiple angles, transform the media into a 3-dimensional representation of the patient's face, and transmit, the 3-dimensional representation of the patient's face to the processing component. The processing component is configured to integrate the 3-dimensional representation of the patient's face with a 3-dimensional representation of the patient's intra-oral scan to visualize a dental treatment plan for the patient.

In another aspect, the processing component is further configured to receive the 3-dimensional representation of the patient's intra-oral scan from a dental scanner.

In another aspect, the processing component is further configured to integrate with one or more dental treatment planning applications to use a visualization of the dental treatment plan.

In another aspect, the at least one device is a mobile device with a built-in camera.

In another aspect, the media includes a plurality of 2-dimensional images of the patient's faced captured by moving the mobile device around the patient's face.

In another aspect, the media includes a video of the patient's face.

In another aspect, the video is captured using a multi-camera system configured to capture images of the patient's face from a plurality of angles at the same time, the at least one device being the multi-camera system.

In another aspect, a result of integrating the 3-dimensional representation of the patient's face with the 3-dimensional representation of the patient's intra-Oral scan can allow an operator of the at least one device to visualize movement of facial tissues, lips, facial expressions once the dental treatment plan is applied to the patient's teeth.

In another aspect, a mobile application on the at least one device is configured to provide real-time guidance for moving the mobile device around the patient's face in order to optimize the media captured.

In another aspect, the processing component is configured to integrate the 3-dimensional representation of the patient's face with the 3-dimensional representation of the patient's intra-oral scan by identifying inner mouth region of the patient in the 3-dimensional representation of the patient's face; removing the inner mouth region from the 3-dimensional representation of the patient's face; determining a scaled-rigid relative transform for the 3-dimensional representation of the patient's face to the 3-dimensional representation of the patient's intra-oral scan; replacing the inner mouth region removed from the 3-dimensional representation with the 3-dimensional representation of the intra-oral scan; and aligning the 3-dimensional representation of the patient's face with the 3-dimensional representation of the patient's intra-oral scan based on the scaled-rigid relative transform.

In another aspect, the inner mouth region is identified in a 2-dimensional space using a machine learning-based model.

In another aspect, the processing component is configured to output a final result of integrating the 3-dimensional representation of the patient's face with the 3-dimensional representation of the patient's intra-oral scan to one or more dental planning tools for further analysis.

In another aspect, a system includes at least one media capturing device; and a processing component (e.g., a cloud-based component) communicatively coupled to the at least one media capturing device. The at least one media capturing device is configured to capture media of a patient's face from at least one angle, transform the media into a 3-dimensional representation of the patient's face, and transmit, the 3-dimensional representation of the patient's face to the processing component. The processing component is configured to integrate the 3-dimensional representation of the patient's face with a 3-dimensional representation of the patient's intra-oral scan to visualize a dental treatment plan for the patient.

In another aspect, the media includes a plurality of 2-dimensional images of the patient's face, and to transform the media into the 3-dimensional representation of the patient's face the device is to: determine a plurality of camera poses, where each camera pose of the plurality of camera poses is determined for a 2-dimensional image of the plurality of 2-dimensional images; generate a plurality of depth maps, where each depth map of the plurality of depth maps is generated for a 2-dimensional image of the plurality of 2-dimensional images based at least in part on the camera pose for the 2-dimensional image; and generate the 3-dimensional representation of the patient's face based on combining information from the plurality of depth maps.

In one aspect, one or more non-transitory computer-readable media includes computer-readable instructions, which when executed by one or more processors of a computing device, cause the computing device to receive, from at least one device, a 3-dimensional representation of the patient's face based on media of the patient's face captured from multiple angles by the at least one device; and integrate, at the computing device, the 3-dimensional representation of the patient's face with a 3-dimensional representation of the patient's intra-oral scan to visualize a dental treatment plan for the patient.

### DESCRIPTION OF THE FIGURES

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1A illustrates an example setting for obtaining a 3-dimensional representation of a patient's face, according to some aspects of the present disclosure;

FIG. 1B illustrates another example setting for obtaining a 3-dimensional representation of a patient's face, according to some aspects of the present disclosure;

FIG. 2 illustrates an example application stored on mobile device of FIG. 1A or a processing component of FIG. 1B for capturing media representing the face of a patient, according to some aspects of the present disclosure;

FIG. 3 illustrates another example screenshot of the application described with reference to FIG. 2 for capturing media representing the face of a patient, according to some aspects of the present disclosure;

FIG. 4 illustrates a 3-dimensional representation of the face of a patient based on media captured using application of FIGS. 2 and 3, according to some aspects of the present disclosure;

FIG. 5 illustrates an example of a system for integrating locally generated 3-dimensional representation of a patient's face with one or more dental treatment plans, according to some aspects of the present disclosure;

FIG. 6 is an example method of capturing 3-dimensional representation of a patient's face, according to some aspects of the present disclosure;

FIG. 7 is an example method of post-processing 3-dimensional representation of a patient's face and fusing the same with 3-dimensional dental treatment plans, according to some aspects of the present disclosure;

FIG. 8A illustrates example outputs of generating 2-dimensional contour, projecting the same on to a surface mesh, and generated a 3-dimensional watertight mesh, according to some aspects of the present disclosure;

FIG. 8B illustrates example output of removing the inner mouth region from the 3-dimensional representation using a 3-dimensional watertight mesh, according to some aspects of the present disclosure;

FIG. 9A illustrates example outputs of scaled-rigid alignment of 3-dimensional representation of the patient's face, after removal of inner mouth region, to a 3-dimensional dental treatment plan, according to some aspects of the present disclosure;

FIG. 9B illustrates an image processing pipeline for generating a 3D image of a face and merging it with one or more 3D models of dental arches, in accordance with an embodiment of the present disclosure;

FIG. 10 illustrates an example neural network that may be utilized for segmentation of 3-dimensional representation of a patient's face for removing inner mouth region, according to some aspects of the present disclosure;

FIG. 11 shows an example computing system, according to some aspects of the present disclosure.

FIG. 12A illustrates a tooth repositioning system including a plurality of appliances, in accordance with embodiments of the present disclosure;

FIG. 12B illustrates a method of orthodontic treatment using a plurality of appliances, in accordance with embodiments;

FIG. 13 illustrates a method for designing an orthodontic appliance to be produced by direct or indirect fabrication, in accordance with embodiments; and

FIG. 14 illustrates a method for digitally planning an orthodontic treatment and/or design or fabrication of an appliance, in accordance with embodiments.

### DETAILED DESCRIPTION OF THE INVENTION

A better understanding of the features and advantages of the present disclosure will be obtained by reference to the following detailed description that sets forth illustrative embodiments, in which the principles of embodiments of the present disclosure are utilized, and the accompanying drawings.

Although the detailed description contains many specifics, these should not be construed as limiting the scope of the disclosure but merely as illustrating different examples and aspects of the present disclosure. It should be appreciated that the scope of the disclosure includes other embodiments not discussed in detail above. Various other modifications, changes and variations which will be apparent to those skilled in the art may be made in the arrangement, operation and details of the methods, systems, and apparatus of the present disclosure provided herein without departing from the spirit and scope of the invention as described herein.

As used herein the terms “dental appliance,” and “tooth receiving appliance” are treated synonymously. As used herein, a “dental positioning appliance” or an “orthodontic appliance” may be treated synonymously and may include any dental appliance configured to change the position of a patient's teeth in accordance with a plan, such as an orthodontic treatment plan. A “dental positioning appliance” or “orthodontic appliance,” as used herein, may include a set of dental appliances configured to incrementally change the position of a patient's teeth over time. As noted herein, dental positioning appliances and/or orthodontic appliances may comprise polymeric appliances configured to move a patient's teeth in accordance with an orthodontic treatment plan.

As used herein the term “and/or” may be used as a functional word to indicate that two words or expressions are to be taken together or individually. For example, the phrase “A and/or B” encompasses A alone, B alone, and A and B together. Depending on context, the term “or” need not exclude one of a plurality of words/expressions. As an example, the phrase “A or B” need not exclude A and B together.

As used herein the terms “torque” and “moment” are treated synonymously.

As used herein a “moment” may encompass a force acting on an object such as a tooth at a distance from a center of resistance. The moment may be calculated with a vector cross product of a vector force applied to a location corresponding to a displacement vector from the center of resistance, for example. The moment may comprise a vector pointing in a direction. A moment opposing another moment may encompass one of the moment vectors oriented toward a first side of the object such as the tooth and the other moment vector oriented toward an opposite side of the object such as tooth, for example. Any discussion herein referring to application of forces on a patient's teeth is equally applicable to application of moments on the teeth, and vice-versa.

As used herein a “plurality of teeth” may encompass two or more teeth. A plurality of teeth may, but need not, comprise adjacent teeth. In some embodiments, one or more posterior teeth comprises one or more of a molar, a premolar or a canine, and one or more anterior teeth comprising one or more of a central incisor, a lateral incisor, a cuspid, a first bicuspid or a second bicuspid.

The embodiments disclosed herein may be well suited for moving one or more teeth of a first group of one or more teeth or moving one or more of a second group of one or more teeth, and combinations thereof.

Example embodiments disclosed herein may be well suited for combination with one or more commercially available tooth moving components such as attachments and polymeric shell appliances (e.g., orthodontic aligners). In some embodiments, the appliance and one or more attachments are configured to move one or more teeth along a tooth movement vector comprising six degrees of freedom, in which three degrees of freedom are rotational and three degrees of freedom are translation.

The present disclosure provides orthodontic appliances and related systems, methods, and devices. Repositioning of teeth may be accomplished with the use of a series of removable elastic positioning appliances such as the Invisalign® system available from Align Technology, Inc., the assignee of the present disclosure. Such appliances may have a thin shell of elastic material that generally conforms to a patient's teeth but is slightly out of alignment with an initial or immediately prior tooth configuration. Placement of the appliance over the teeth applies controlled forces in specific locations to gradually move the teeth into the new configuration. Repetition of this process with successive appliances comprising new configurations eventually moves the teeth through a series of intermediate configurations or alignment patterns to a final desired configuration. Repositioning of teeth may be accomplished through other series of removable orthodontic and/or dental appliances, including polymeric shell appliances.

Although reference is made to an appliance comprising a polymeric shell appliance, the embodiments disclosed herein are well suited for use with many appliances that receive teeth, for example appliances without one or more of polymers or shells. The appliance can be fabricated with one or more of many materials such as metal, glass, reinforced fibers, carbon fiber, composites, reinforced composites, aluminum, biological materials, and combinations thereof for example. The appliance can be shaped in many ways, such as with thermoforming or direct fabrication as described herein, for example. Alternatively, or in combination, the appliance can be fabricated with machining such as an appliance fabricated from a block of material with computer numeric control machining. Additionally, though reference is made herein to orthodontic appliances, at least some of the techniques described herein may apply to restorative and/or other dental appliances, including without limitation crowns, veneers, teeth-whitening appliances, teeth-protective appliances, etc.

In the design of virtual representations of living beings, an “uncanny valley” can relate to the extent a virtual object's resemblance to a living being corresponds to emotional responses to the virtual object. The concept of an uncanny valley may suggest humanoid virtual objects which appear almost, but not exactly, like real human beings (robots, 3D computer animations, lifelike dolls, etc.) elicit uncanny, or strangely familiar, feelings of eeriness and revulsion in observers. A virtual object that appears “almost” human risks may elicit cold, eerie, and/or other non-emotional feelings in viewers.

In the context of treatment planning, the uncanny valley problem may cause people to negatively react to humanoid representations of themselves. As an example, people viewing a 3D virtual representation of themselves after an orthodontic treatment plan may be confronted with an unfamiliar, robotic, or non-humanoid view of themselves.

Systems and methods that reduce the uncanny valley reaction and take into account the relationships between the positions and orientations of teeth and the shape and position of facial features of a patient could help in increasing the effectiveness and acceptance of orthodontic treatment, particularly orthodontic treatments involving virtual representations and/or virtual 3D models of living beings before, during and/or after the application of orthodontic treatment plans.

The present disclosure provides tools for capturing a 3-dimensional representation of a patient's face across multiple angles (e.g., using a mobile device). The 3-dimensional representation may then be integrated (fused) with at least a 3-dimensional intra-oral mesh of the patient's teeth. The resulting high resolution combined representation may then be integrated with one or more 3-dimensional treatment planning products, that can provide accurate and realistic results of a dental treatment plan for the patient.

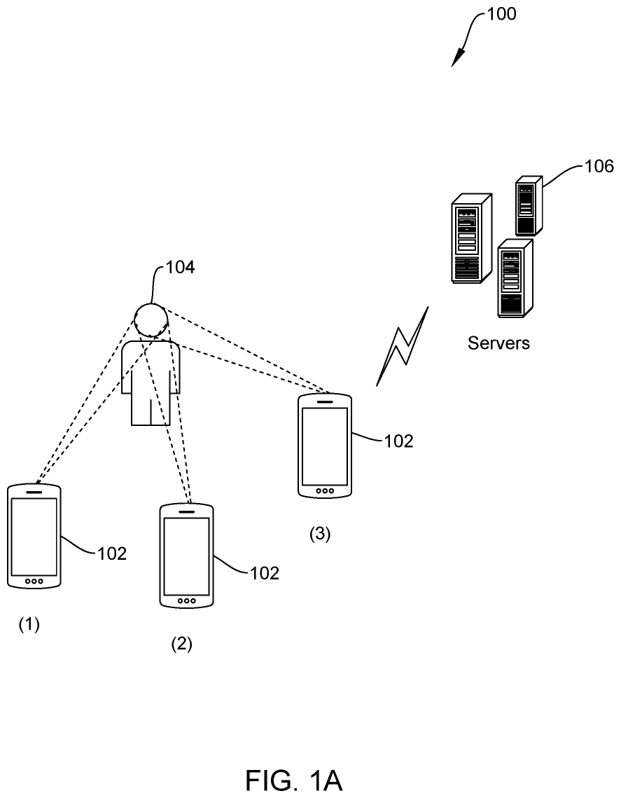

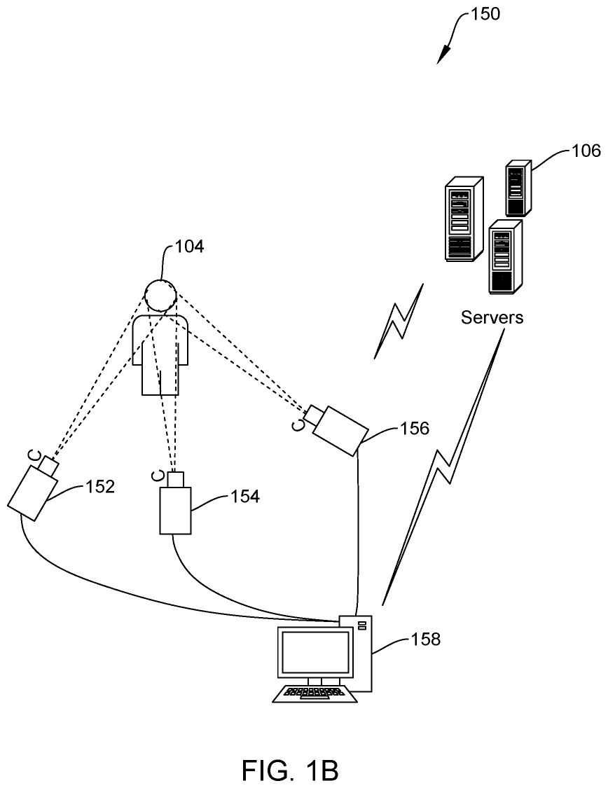

FIG. 1A illustrates an example setting for obtaining a 3-dimensional representation of a patient's face, according to some aspects of the present disclosure. Setting 100 can be in a physical location (e.g., a dentist's office, a patient's home, or any other location). A mobile device 102 may be used to obtain media of a face of patient 104. Mobile device 102 may be any known or to be developed consumer electronic device equipped with media capturing components (e.g., built-in media capturing components such as a camera for capturing 2-dimensional photographs and/or videos). Such mobile device 102 may also be capable of having one or more applications installed thereon, which as will be described below, can be used to transform the captured media into a 3-dimensional representation of the face of patient 104 in a very short order on mobile device 102 itself. Mobile device 102 may also be equipped with one or more electronic components enabling mobile device 102 to establish wired and/or wireless communication with one or more remote components and/or the Internet. The one or more remote components may be cloud-accessible or cloud-based processing components and/or servers such as servers 106 (the use of which within the context of the present disclosure will be further described below with reference to subsequent figures. Cloud-based processing refers to computing that relies on remote servers (typically located in data centers) for processing power, rather than using local servers or personal devices. Cloud-based processing generally relies on virtual machines (VMs), which emulate a physical computer, enabling users to run applications on cloud servers as if they were running on local machines. Other options for cloud-based processing are use of containers, which are lightweight alternatives to VMs that encapsulate an application with its dependencies, libraries, and binaries in one package. This ensures that the application will run consistently across different environments. Cloud-based processing may also take advantage of serverless computing, in which a cloud provider dynamically manages the allocation of machine resources.

Non-limiting examples of mobile device 102 include, but are not limited to, a smart phone (e.g., Apple iPhone, Samsung Galaxy, etc.), a tablet (e.g., Apple iPad, Microsoft Tablet), and/or any other handheld electronic device having the capabilities described above.

As shown in FIG. 1, mobile device 102 may be moved around to capture media of the face of patient 104 from different angles. In the non-limiting example of FIG. 1, three different positions (1), (2), and (3) are shown, each corresponding to a different angle at which mobile device 102 can capture media representing the face of patient 104. However, the present disclosure is not limited thereto and more or less media of the face of patient 104 may be captured at different angles. As mentioned, the captured media may include 2-dimensional photographs (e.g., images) and/or videos. Throughout the present disclosure photographs may be referred to as one or more still (static) images taken using one or more cameras (e.g., built-in camera of mobile device 102 or several interconnected cameras). In such photographs, static variations in facial expressions of patient 104 (e.g., variations in tissue movement and articulation) may be captured. Video(s), in this disclosure, may refer to a series of still and/or moving images of patient 104 taken using one or multiple cameras (that may be communicatively coupled to mobile device 102 and/or servers 106) at the same time such that dynamic variations in facial expressions of patient 104 (e.g., variations in tissue movement and articulation) may be captured.

In one example, the movement of mobile device 102 may be continuous (without a pause) as mobile device 102 moves between positions (1), (2), and (3) or may involve a short pause of mobile 102 at each angle for capturing the media.

FIG. 1B illustrates another example setting for obtaining a 3-dimensional representation of a patient's face, according to some aspects of the present disclosure. Example setting 150 of FIG. 1B differs from the example setting 100 of FIG. 1A in that instead of a mobile device being moved around patient 104's face for capturing media of patient 104's face from different angles, a stationary set of cameras 152, 154, and 156 (multi-camera system) may be used. While FIG. 1B illustrates the use of only three cameras, the present disclosure is not limited thereto, and the number of cameras can be two or more.

In one example, each of cameras 152, 154, and 156 may come in pairs (e.g., a pair of cameras 152, a pair of cameras 154, and a pair of cameras 156). Cameras in pairs may be used to capture stereo and/or 3-dimensional images, video and/or effects.

Cameras 152, 154, and 156 may be used to capture media of patient 104's face in the form of a videos (as will be defined below). Cameras 152, 154, and 156 may be communicatively coupled to a processing component such as desktop 158 or may directly be connected to server 106. Generation of 3-dimensional representations of patient 104's face may then take place on desktop 158 or on servers 106.

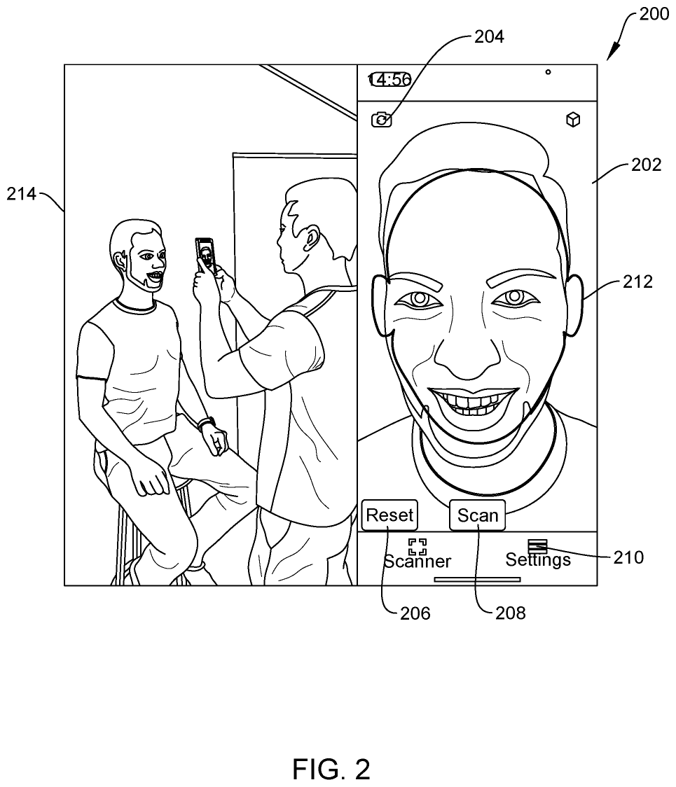

FIG. 2 illustrates a user interface for an example application stored on mobile device 102 of FIG. 1 for capturing media representing the face of a patient, according to some aspects of the present disclosure. Split screen 200 of FIG. 2 includes a snapshot of an application 202 (downloadable and executable on mobile device 102 of FIG. 1). A user of mobile device 102 may select application 202 from multiple available applications. Application 202 may have a number of options displayed thereon (e.g., option 204 for selecting one of available cameras on mobile device 102 such as front or back camera, reset button 206 for restarting the media capturing process whenever applicable, scan button 208 to start the media capturing process, hamburger menu 210 for accessing various other options for application 202 such as settings, privacy notifications, media capturing settings, prior recordings, etc.). In one example, scan button 208 may change to stop button such that when selected, after scan button 208 is selected, the media capturing process stops.

Application 202, once the scan button 208 is selected, may start capturing media of the face of patient 104 with identified contour 212.

Split screen 200 also shows a snapshot 214 of using mobile device 102 and application 202 for capturing media representing the face of patient 104, as described above with reference to FIG. 1.

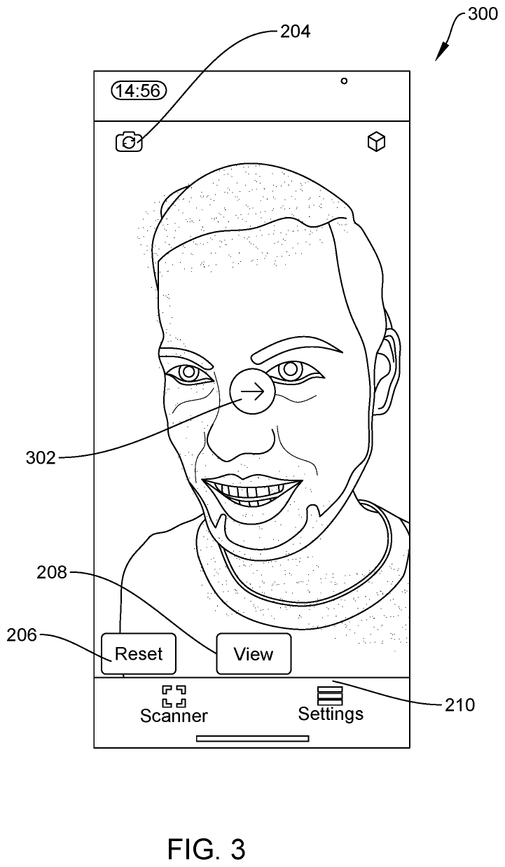

FIG. 3 illustrates another example screenshot of the application described with reference to FIG. 2 for capturing media representing the face of a patient, according to some aspects of the present disclosure. Example screenshot 300 is the same as screenshot 202 of FIG. 2 except that it illustrates an additional feature of application 202. This additional feature is a live media capturing guidance 302. As a user of mobile device 102 moves around the patient's face to capture the media of the face, live media capturing guidance 302 may guide the user on how best to move mobile device 102 (e.g., left, right, up, and/or down) so as to optimize the media captured. In one example, the direction of the arrow within live media capturing guidance 302 may change depending on the recommended movement direction (e.g., may point up if the recommendation is for the user to move mobile device 102 up, may point left if the recommendation is for the user to move mobile device 102 left, etc.).

Once media of the face of a patient is captured using mobile device 102 and application 202 (which can be, for example, Bellus3d or ARKit's face reconstruction application), a 3-dimensional representation of the patient's face may be constructed and displayed within application 202. This transformation of 2-dimensional photographs/images and/or videos into a 3-dimensional representation of the face is performed locally on mobile device 102 and without resorting to use of any external resources or computational capabilities such as servers 106. This transformation may result in a high resolution 3-dimensional representation in a relatively short period of time (e.g., seconds or minutes). The transformation of images and videos into the 3-dimensional representation may be performed according to any known or to be developed signal and image processing technique for generating 3-dimensional representation of objects.

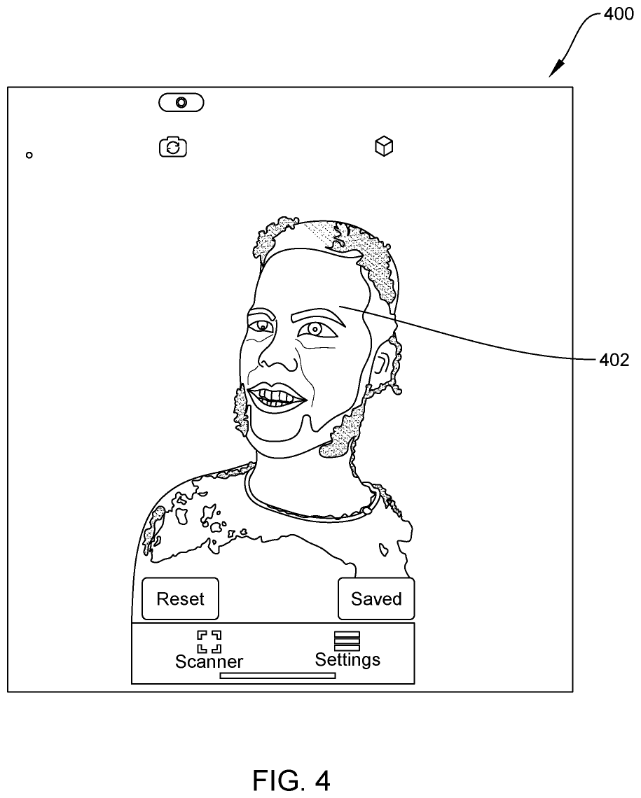

FIG. 4 illustrates a 3-dimensional representation of the face of a patient based on media captured using application of FIGS. 2 and 3, according to some aspects of the present disclosure. Snapshot 400 shows 3-dimensional representation 402 of the face of patient 104 whose media was taken using application 202 installed on mobile device 102, as described above with reference to FIGS. 1-3. In one example, 3-dimensional representation 402 may be interactive such that using an input (e.g., a touch input or any other form of input), a user of mobile device 102 can move 3-dimensional representation 402 around (e.g., up, down, left, right, and/or rotate the same around), zoom in and out, and view 3-dimensional representation 402 from different angles (e.g., by rotating the 3-dimensional representation).

Thereafter, 3-dimensional representation 402 may be transmitted from mobile device 102 to servers 106 for further processing and integration with one or more dental treatment plans, as will be further described below, in order to provide a doctor and/or the patient a complete (holistic) and realistic representation of what the dental treatment plan would look like once completed.

In one or more aspects, when the captured media is a video from different angles, the holistic representation would enable the doctor and patient to see the effect of various facial movements once the dental treatment plan is implemented (e.g., visualize what the patient's smile would look like after the dental treatment plan is implemented, how movement of lips, mouth, tongue, and other facial tissues and elements such as eyes and cheeks would affect the appearance of the patient once the dental treatment plan is applied, etc.).

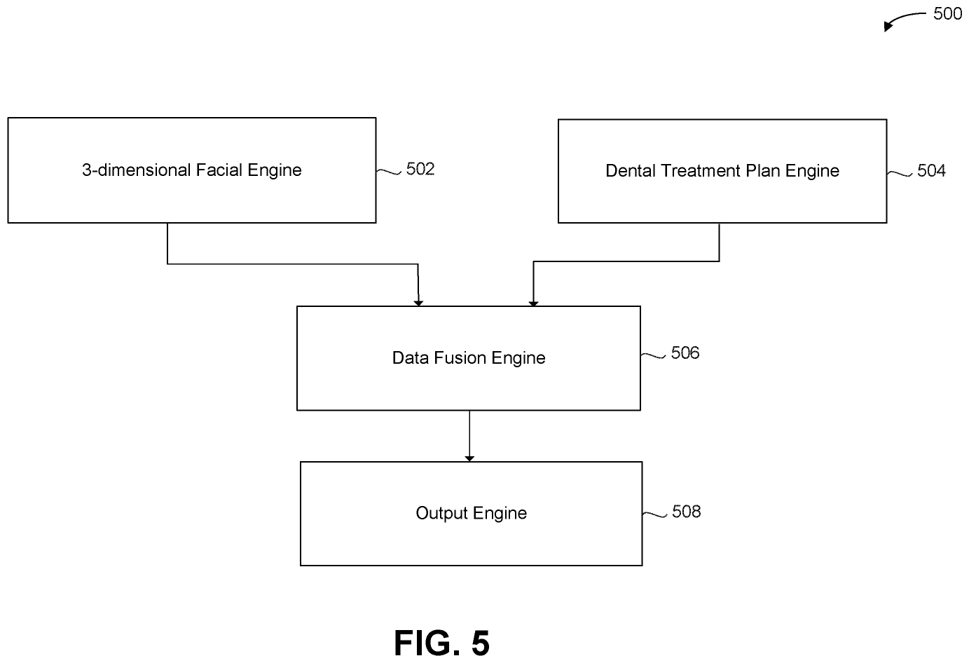

FIG. 5 illustrates an example of a system for integrating a locally generated 3-dimensional representation of a patient's face with one or more dental treatment plans, according to some aspects of the present disclosure. System 500 of FIG. 5 illustrates a number of engines 502-508. Each engine may correspond to a set of computer-readable instructions stored on one or more memories and executed by one or more processors to implement a set of functionalities for fusing a 3-dimensional representation of a patient's face with one or more dental treatment plans (e.g., with 3-dimensional models of the patient's upper and/or lower dental arches) in order to generate a visualization of the ultimate facial appearance of the patient after the dental treatment plan is implemented. Non-limiting examples of agents or engines shown in FIG. 5 may be implemented at one or more servers such as servers 106 of FIG. 1. While shown as separate logical engines in FIG. 5, a single processor may implement the functionalities of two or more engines forming system 500.

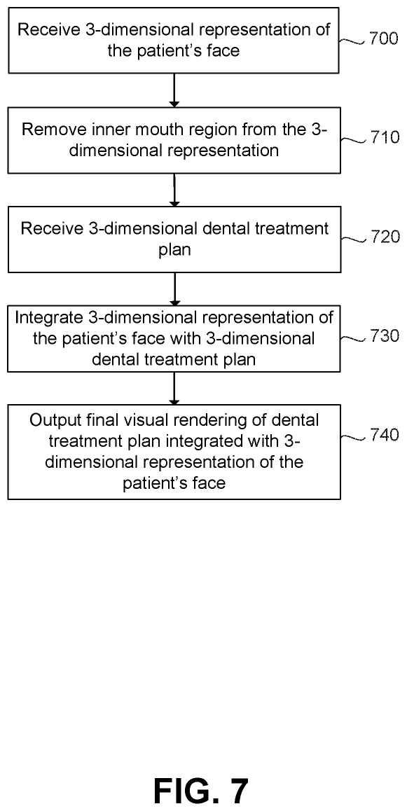

System 500 may include a 3-dimensional facial engine 502, dental treatment plan engine 504, data fusion engine 506, and output engine 508. In one example, 3-dimensional facial engine 502 may receive 3-dimensional representation 402 of the face of patient 104 generated on mobile device 102, from mobile device 102. Then, 3-dimensional facial engine 502 may perform a series of image segmentation and processing steps to remove inner mouth section of patient 104 from 3-dimensional representation 402 in order to be replaced with a 3-dimensional representation of a dental treatment plan provided by dental treatment plan engine 504. 3-dimensional facial engine 502 may perform any other functionalities related to modifying and/or enhancing 3-dimensional representation 402. This process will be further described below with reference to FIG. 7.

Dental treatment plan engine 504 may be interfaced with one or more existing 3-dimensional dental planning software and/or tools including, but not limited to, software and tools developed by Align Technology, Inc. of San Jose, CA. Such non-limiting tools can include a dental or intraoral scanner, Clincheck®, iTero®, and Exocad® applications developed and marketed by Align Technology, Inc. of San Jose, CA. While these are named as exemplary 3-dimensional dental planning tools, the present disclosure is not limited to these and any other known or to be developed 3-dimensional dental planning tool may be integrated with/communicatively coupled to dental treatment planning engine 504 to receive a 3-dimensional dental treatment plan to be fused with 3-dimensional representation 402.

Fusion engine 506 may perform a number of image processing techniques to fuse (combine/integrate) 3-dimensional representation 402 (after removing inner mouth region of 3-dimensional representation 402) with a 3-dimensional dental treatment plan received from dental treatment planning engine 504. This fusion process will be further described below with reference to FIG. 7. A 3-dimensional dental treatment plan may also be referred to and/or may include one or more 3-dimensional intra-oral scans of the patient that can be obtained using, for example, the iTero® tool.

Once the fusion process is complete, output engine 508 may output a final 3-dimensional representation 402 modified to incorporate a 3-dimensional dental treatment plan (e.g., one or more 3-dimensional models of dental arches of a patient at one or more stages of orthodontic treatment) that would allow a dentist, a medical professional, and/or the patient to visualize the look and feel of the dental treatment plan on their facial appearance once implemented.

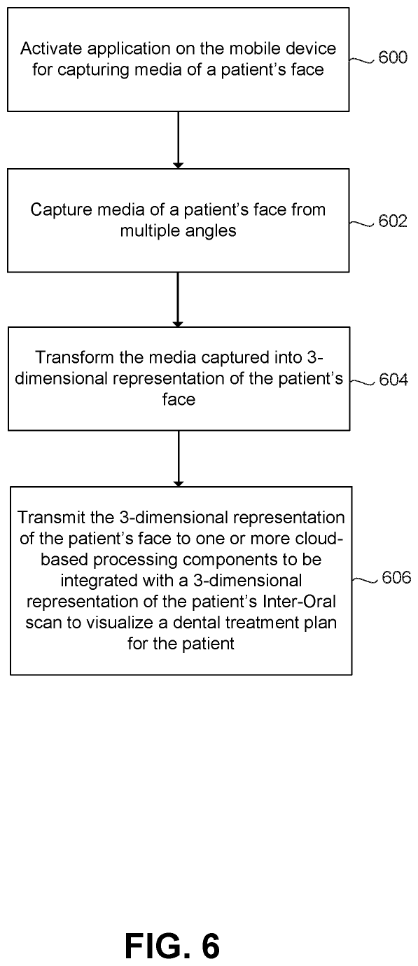

FIG. 6 is an example method of capturing a 3-dimensional representation of a patient's face, according to some aspects of the present disclosure. The process of FIG. 6 will be described from the perspective of mobile device 102 of FIG. 1. However, it should be noted that mobile device 102 may have one or more memories having computer-readable instructions of application 202 stored therein, which when executed by one or more processors on mobile device 102, enable mobile device 102 to implement the steps of FIG. 6. In describing FIG. 6 references may be made to one or more of FIGS. 1-5.

At step 600, mobile device 102 may activate application 202. This activation may be triggered by a user of mobile device 102 opening application 202 and/or selecting scan button 208 as described above with reference to FIG. 2.

At step 602, mobile device 102 may capture media of patient 104 from multiple angles (e.g., positions (1), (2), and (3) described above with reference to FIG. 1). As described, the media captured can be one or more still images/photographs of the head and face of patient 104 or could be a video of the head and face of patient 104. Throughout this disclosure, any reference to a patient's face may also include the head of the patient as well. The media may be captured by a built-in camera of mobile device 102 and/or an external media capturing devices coupled (physically or communicatively) to mobile device 102.

As noted above, application 202 may provide real-time media capturing guidance 302, which may guide the user of mobile device 102 to move mobile device 102 in different directions as it moves around patient 104 in order to optimize the media captured for purposes of transforming the captured media into 3-dimensional representation 402.

At step 604, mobile device 102 may transform the media captured at step 602 into 3-dimensional representation 402 of the head and face of patient 104, as described above with reference to FIGS. 3 and 4.

At step 606, mobile device 102 may transmit 3-dimensional representation 402 to one or more servers 106 for further processing and fusion with dental treatment plans (one or more 3-dimensional intra-oral scans for patient 104), which will be further described below with reference to FIG. 7.

FIG. 7 is an example method of post-processing a 3-dimensional representation of a patient's face and fusing the same with 3-dimensional dental treatment plans (e.g., with one or more 3-dimensional models of a patient's upper and/or lower dental arches at one or more stages of treatment), according to some aspects of the present disclosure. The process of FIG. 7 will be described from perspective of one of servers 106 of FIG. 1. However, it should be noted that when utilizing more than one server 106, any one or more of servers 106 may perform the steps of FIG. 7. Moreover, it should be noted that such server(s) may have one or more memories having computer-readable instructions stored therein that correspond to logics associated with each of engines 502, 504, 506, and 508 described above with reference to FIG. 5, which when executed by one or more processors, enable server(s) 106 to implement the steps of FIG. 7. In describing FIG. 7, references may be made to one or more of FIGS. 1-6.

At step 700, server 106 (e.g., a cloud-based processing component) may receive 3-dimensional representation 402 of face of patient 104 from mobile device 102.

At step 710, server 106 may implement logics associated with 3-dimensional facial engine 502 to remove at least one element or component from 3-dimensional representation 402. In one example, such element or component can be inner mouth region of patient 104. In the non-limiting example of removing inner-mouth region of patient 104 from 3-dimensional representation 402, the following steps may be taken.

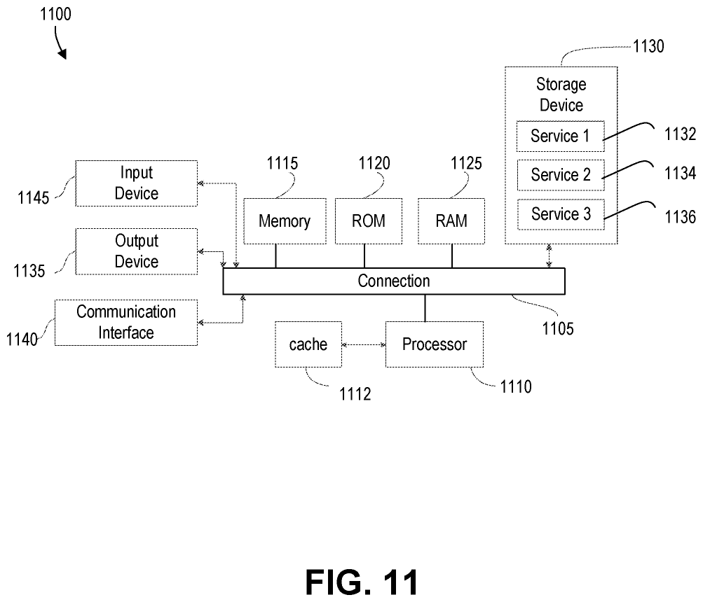

First, 2-dimensional positions of an inner mouth contour may be generated or determined. In one example, one or more trained neural networks (a machine learning process) may be applied to generate or determine the contour. For example, a 3-dimensional representation of the face of patient 104 on a plane from a pre-defined camera projection matrix may be generated. Then an image with the projected 3D representation of the patient's face may be obtained. Using the image, a 2-dimensional inner-mouth segmentation network is executed to define a region that corresponds to the patient's inner mouth. An image processing technique may then be applied to generate contour points that define the inner mouth region. The inner mouth contour points may then be projected onto the 3-dimensional representation given that the projection matrix is known, resulting in the 3-dimensional representation of the patient's face with embedded contours of the patient's inner mouth. This will be further described below with reference to FIG. 11.

Details of training one or more neural networks to generate the inner mouth contour will be described below with reference to FIG. 11.

Thereafter, the generated inner mouth contour is projected onto a surface mesh to generate a 3-dimensional watertight mesh.

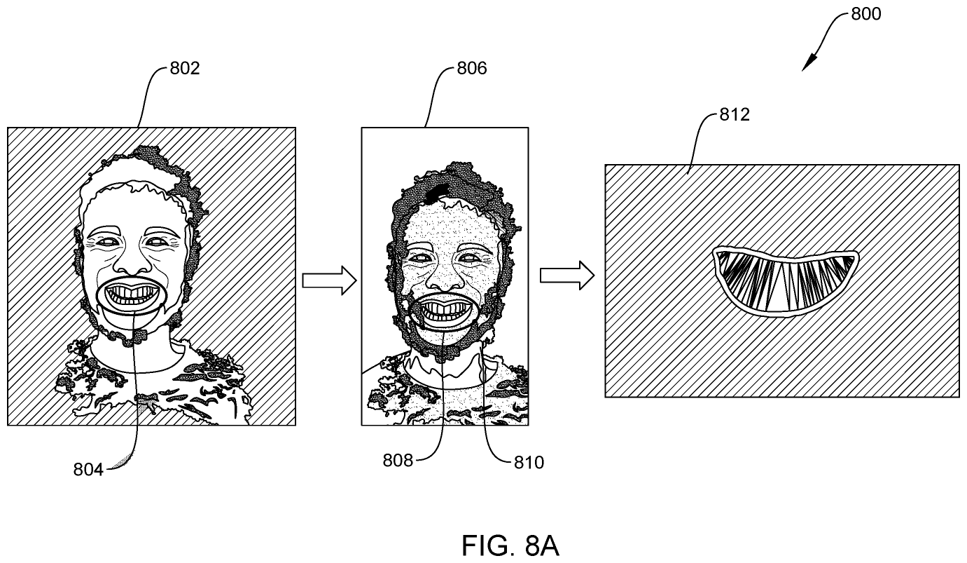

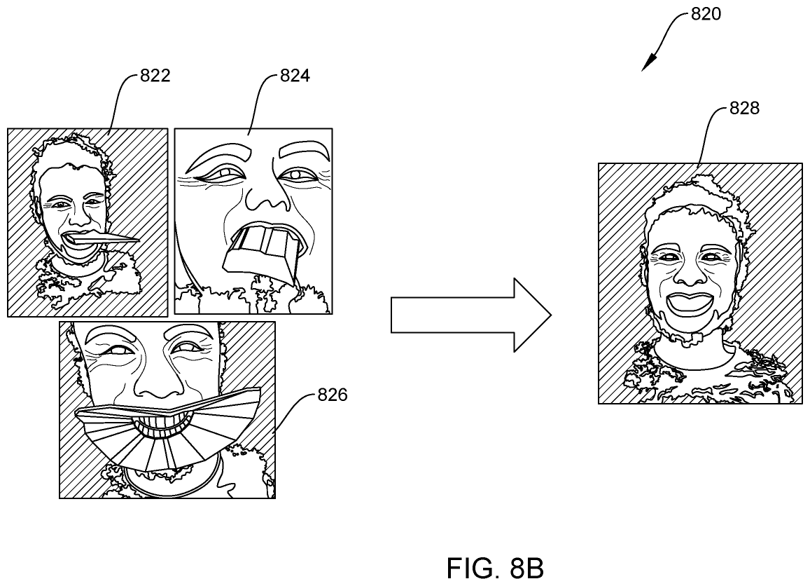

FIG. 8A illustrates example outputs of generating a 2-dimensional contour, projecting the same on to a surface mesh, and generating a 3-dimensional mesh (e.g., a watertight mesh), according to some aspects of the present disclosure. Example 800 includes a snapshot 802 that shows 3-dimensional representation 402 described above and the example inner mouth region 804 to be removed.

Example output 806 shows the result of applying a trained neural network to generate 2-dimensional inner mouth contour 808 that is then projected onto a surface mesh 810.

Example output 812 shows the generated 3-dimensional watertight mesh described above.

Referring back to FIG. 7, once the 3-dimensional watertight mesh is generated, a 3-dimensional geometry processing algorithm may be applied to the cut 3-dimensional representation 402 with the 3-dimensional watertight mesh. Any known or to be developed 3-dimensional geometry processing algorithm may be applied for cutting 3-dimensional representation 402 with the 3-dimensional watertight mesh described above.

FIG. 8B illustrates example output of removing the inner mouth region from the 3-dimensional representation using a 3-dimensional watertight mesh, according to some aspects of the present disclosure. In example 820 of FIG. 8B, outputs 822, 824, and 826 illustrate the process of cutting 3-dimensional representation 402 with 3-dimensional watertight mesh and output 828 illustrates a final result of 3-dimensional representation 402 with the inner mouth region removed.

Referring back to FIG. 7, with inner mouth region of 3-dimensional representation 402 removed at step 710, at step 720, server 106 may receive a 3-dimensional dental treatment plan. As described above, dental treatment planning engine 504 may be integrated (communicatively coupled to) one or more dental planning tools to receive a 3-dimensional representation of a dental treatment plan for patient 104. Such 3-dimensional representation may visualize the resulting look and feel of patient 104's teeth after the dental treatment plan is implemented.

At step 730, server 106 may integrate 3-dimensional representation 402, after removing the inner mouth region, with 3-dimensional dental treatment plan received at step 720. This integration process may be as follows. In one example, server 106 may perform a scaled-rigid alignment of 3-dimensional representation 402 to 3-dimensional dental treatment plan (e.g., to one or more 3D models of dental arches of a dental treatment plan) received at step 730. This scaled-rigid alignment may be based on several poses of patient 104 captured by mobile device 102, 2-dimensional machine learning segmentation described above, and a segmented intra-oral scan. Server 106 may have access to several poses of patient 104 (e.g., raw captured media) as mobile device 102 may also transmit the raw captured media to server 106 together with 3-dimensional representation 402.

In one example, such scaled-rigid alignment may include server 106 determining (computing) a scaled-rigid relative transform between 3-dimensional representation 402 and a 3D model of one or more dental arches of patient 104 and/or of one or more 3-dimensional intra-oral scans of patient 104 using any known or to be developed method.

Thereafter, server 106 places a 3-dimensional intra-oral scan or a 3D model of an upper and/or lower dental arch (or a portion thereof) in the removed inner mouth region within 3-dimensional representation 402 (i.e., within output 828 of FIG. 8B). Once placed therein, server 106 may perform a scaled-rigid alignment process to align the 3-dimensional intra-oral scan or one or more 3D models of the upper and/or lower dental arches within 3-dimensional representation 402 and scale 3-dimensional representation 402 such that relative sizes of patient 104's face and 3-dimensional intra-oral scan are proportional to each other and do not appear unrealistic.

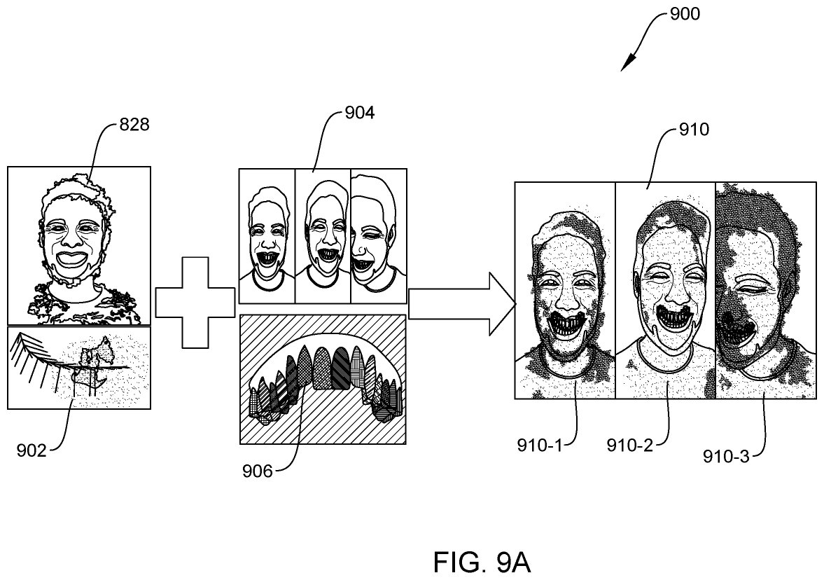

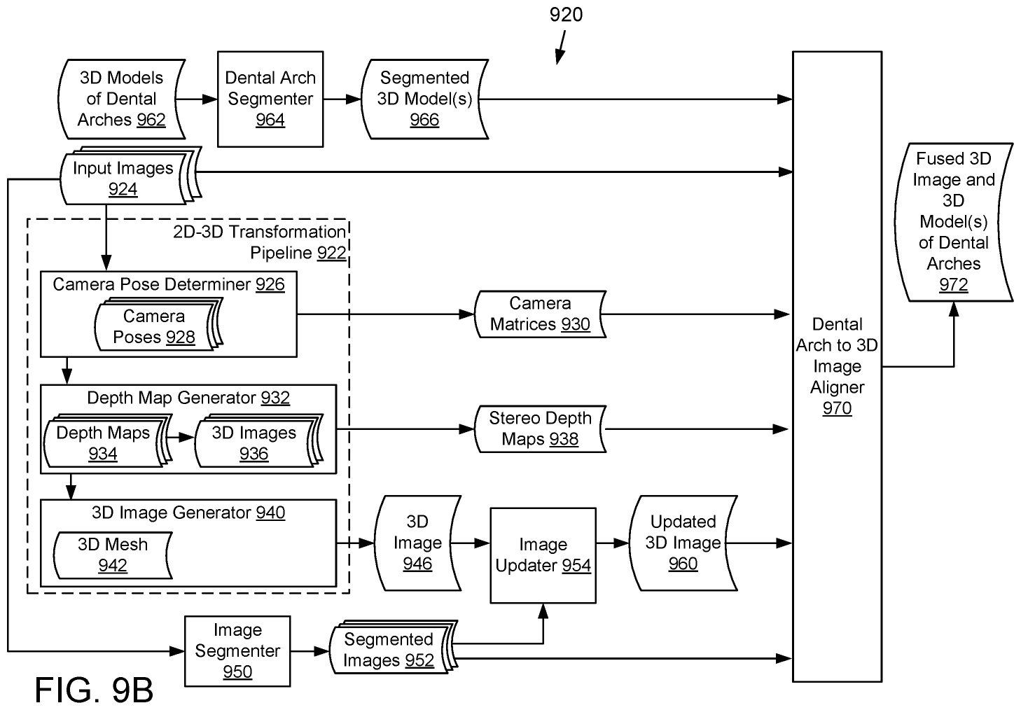

FIG. 9A illustrates example outputs of scaled-rigid alignment of 3-dimensional representation of the patient's face, after removal of inner mouth region, to a 3-dimensional dental treatment plan (e.g., to one or more 3D models of dental arches of a patient at a stage of treatment), according to some aspects of the present disclosure. Example 900 of FIG. 9 includes several outputs including 3-dimensional representation 828 of patient 104 with inner mouth removed as described above with reference to FIG. 8B. 3-dimensional representation 828 may then be aligned with 3-dimensional dental plan (e.g., with a 3D model of an upper and/or lower dental arch at a stage of treatment) based on several poses of patient 104 (captured in output 902) and 2-dimensional machine learning based segmentation of inner mouth region as described with reference to FIG. 8A above (illustrated as output 904 in FIG. 9) and segmentation of 3-dimensional intra-oral scan or 3D model of a dental arch 906 in FIG. 9. A resulting integration and alignment is shown as output 910 in FIG. 9. Output 910 includes three example snapshots 910-1, 910-2, and 910-3 illustrating gradual adjustments to obtain alignment of 3-dimensional intra-oral scan or 3D model of a dental arch 906 with 3-dimensional representation 402 of patient 104's face.

Referring back to FIG. 7 and after integration of 3-dimensional intra-oral scan or 3D model of a dental arch with 3-dimensional representation of patient 104's face is complete, at step 740, server 106 may output the final result that provides a visualization of patient 104's face with the dental treatment plan applied.

As noted above, server 106 may be coupled with one or more dental planning tools named above. Accordingly, the final result may be output to any one such planning tool to be further utilized by a dentist, a hygienist, a dental technician, and/or patient 104 to determine sufficiency of a proposed dental treatment plan and perform any modifications and/or adjustments thereto, as needed.

FIG. 9B illustrates an image processing pipeline 220 for generating a 3D image (e.g., a 3-dimensional representation) of a face and merging it with one or more 3D models of dental arches (e.g., a 3-dimensional representation of the patient's intra-oral scan), in accordance with an embodiment of the present disclosure. The image processing pipeline 220 can be conceptually divided into a 2D to 3D transformation pipeline 922 and a 3D image fusion pipeline (not called out). FIG. 9B is described with reference to images, but works equally well with video (e.g., frames of a video).

The 2D-3D transformation pipeline 922 may include a camera pose determiner 926 that may perform tracking of camera poses for input images 924. Camera pose determiner 926 may receive a plurality of input 2D images 924 generated from different views corresponding to different camera positions and/or orientations (referred to as camera poses). The camera pose determiner 926 may estimate a camera pose 928 for each of the input images. Each camera pose 928 may include, for example, an x, y, z position and/or an x, y, z orientation (e.g., angle(s) relative to a global x, y, z axis). Each camera pose 928 may represent a position in space and an orientation in space of a camera that generated an input image 924. Camera poses 928 may be determined based on identifying 2D features in input images 924, matching 2D features between images, and determining key pairs for frames from the 2D feature matching. Adjustments between images can be determined from the 2D feature mapping, and adjustments may be bundled to estimate camera poses, and thus ultimately the 3D locations of 3D features. Camera pose determiner 926 may generate one or more camera matrices 928 that include camera poses 928 for multiple input images 924 as well as camera parameters (e.g., extrinsic and/or intrinsic camera parameters). Intrinsic camera parameters are internal camera parameters, and they don't change regardless of the scene being captured. They relate to the camera's internal characteristics. Examples of intrinsic parameters include focal length (the distance between the camera's center of projection (usually the center of the lens) and the image plane (sensor)), optical center or principal point (the point on the image plane to which rays parallel to the optical axis converge), skew values (describes the angle between the pixel axes), and/or lens distortion parameters. Extrinsic parameters are parameters that describe the position and orientation of the camera in the world coordinate system. They are external to the camera and define its location relative to a world frame of reference. Extrinsic parameters may include, for example, a rotation matrix (e.g., a 3×3 matrix that captures the orientation of the camera in the world and relates the coordinates of a point in the camera frame to its coordinates in the world frame) and/or a translation vector (e.g., a 3×1 matrix that represents the position of the camera's origin in world coordinates).

Depth map generator 932 receives the camera poses 928 and/or camera matrices 930 from camera pose determiner 926, and uses this information to estimate a depth map 934 for each of the input images 924. A depth map is a 2D representation of the depth information of a scene. For every pixel in the depth map, the value (often a grayscale value) indicates the distance between the viewpoint (typically a camera or viewer) and the corresponding point in the real-world scene. The estimated depth maps 934 of two or more images may be fused to generate fused or stereo depth maps 938. When two cameras (or two images generated by the same camera from different viewpoints) capture the same scene from slightly different viewpoints, the difference in the apparent position of an object in the two images (called disparity) can be used to compute the object's depth. Stereo depth maps may be based on pairs of images, and may be more accurate that depth maps generated from a single image.

3D image generator 940 receives the stereo depth maps 938 from depth map generator 932, and uses this information to generate a 3D mesh 942 by combining point clouds from the depth maps 934 and/or stereo depth maps 938 and the camera poses 938 associated with those depth maps or stereo depth maps. The 3D mesh 942 lacks color or texture information, but provides a 3D model or image generated from the depth information contained in the stereo depth maps 938. The 3D image generator 940 then applies texturing to the generated 3D mesh 942 based on the color information contained in the input images 924, and generates a 3D image 946 or model of the face that combines information from the multiple 2D input images 924.

In some instances, the mouth region in the 3D image 946 may not have a sufficiently high quality for clinical purposes. Teeth may be highly reflective, which may cause a geometry of the inner mouth region to be inaccurate.

Once the 3D image 946 of a face of a patient has been generated, it can be beneficial to merge the 3D image of the face with 3D models of dental arches of the patient, as described elsewhere herein. In one embodiment, an image segmenter 950 receives one or more input images 924 and segments at least a mouth region of the one or more input images 924 to generate segmented images 952 including segmentation information for the inner mouth region (e.g., in which each of the teeth in the image have been identified and/or labeled. Image segmentation is a computer vision task where an image is partitioned into multiple segments or regions, each representing different objects or parts of objects. Segmentation may be performed, for example, using one or more trained machine learning models (e.g., one or more neural networks). The one or more neural networks may receive as input a 2D image of a face, and may output segmentation information segmenting the 2D image into a mouth region and a non-mouth region, and optionally further segmenting the mouth region into individual teeth and/or gingiva. This may be performed for each input image 924 or for only a subset of input images 924. Depending on the architecture, the output of the machine learning model might either be a label for each pixel (semantic segmentation) or a label for each distinct object instance (instance segmentation).

The segmented images 952 may be projected into 3D using the appropriate camera poses 928, depth maps 934 and/or stereo depth maps 938. Based on the projection of the segmented images 952 into 3D, the segmentation information for the inner mouth area may be determined in the 3D image 946. Image updater 954 may update the 3D image by removing the data for the inner mouth region based on the 3D inner mouth segmentation information (e.g., including 3D teeth segmentation). Updated 3D image 960 may be generated, in which the data for the inner mouth region has been removed or deleted.

One or more 3D models of patient dental arches 962 (e.g., a 3D model of the patient's upper dental arch and a 3D model of the patient's lower dental arch) may have been generated based on intraoral scanning. In embodiments, a dental arch segmenter 964 segments the 3D models of the dental arches to generate segmented 3D models 966. Segmentation may be performed, for example, using one or more trained machine learning models (e.g., one or more neural networks). The one or more neural networks may receive as input a 3D model of a dental arch and/or projections of a 3D model of a dental arch onto one or more 2D planes, and may output segmentation information segmenting the 3D model into individual teeth and/or gingiva. This may be performed for both the upper and lower dental arch 3D models.

In embodiments, segmented images 952, input images 924, camera matrices 930, and/or stereo depth maps 938 are input into a dental arch to 3D image aligner 970. Additionally, one or more segmented 3D models 966 may also be input into dental arch to 3D image aligner 970. Dental arch to 3D image aligner 970 may perform a multi-view face alignment, in which two or more of the segmented 2D images 952 are aligned with (e.g., registered with) the segmented 3D model(s) 966. For example, for each segmented image 952 one or more transformations may be computed that may adjust sizing (e.g., scale), x, y and/or z position, and/or rotation about x, y and/or z axes to register the segmented image 952 (e.g., the segmented mouth region of the segmented image) to the segmented 3D model. Based on registrations/alignment of multiple segmented images 952 to the segmented 3D model(s), a minimization or optimization problem may be solved to determine a single set of transformation parameters that can be applied to the updated 3D image 960 to register the updated 3D image 960 to the segmented 3D model(s) 966 (or to the 3D models of the dental arch(es) 962). The single set of transformation parameters may provide a rigid transform of the teeth relative to the camera poses of each of the respective images that may be applied to the respective images to align the respective images with the 3D model(s). That rigid transformation could be used to fit all of the different images to the 3D model(s) of the dental arches.

Once the rigid transformation is determined, dental arch to 3D image aligner 970 may apply the determined rigid transformation to merge the 3D model(s) of the dental arch(es) 962 (or the segmented 3D models 966) to the updated 3D image 960. Since the inner mouth region was removed in the updated 3D image 960, the inner mouth region would be filled in by information from the 3D model(s) of the dental arch(es) 962. Dental arch to 3D image aligner 970 may output a fused 3D image and 3D model(s) of dental arches 972, which may be output to a display.

Accordingly, from multi-view 2D images of a patient's face, processing logic generates a 3D representation of the patient's facial structure and texture based on estimates of camera pose (e.g., camera matrices), depth maps, and/or other information. Processing logic additionally registers the patient's 3D intra-oral scan data (e.g., 3D models of upper and/or lower dental arches) to the same set of multi-view 2D images. A rigid transform is estimated, which is relative to the estimated camera matrices. This allows processing logic to estimate the rigid transform to align the 3D Face to the 3D intra-oral scan data (e.g., 3D models of upper and/or lower dental arches).

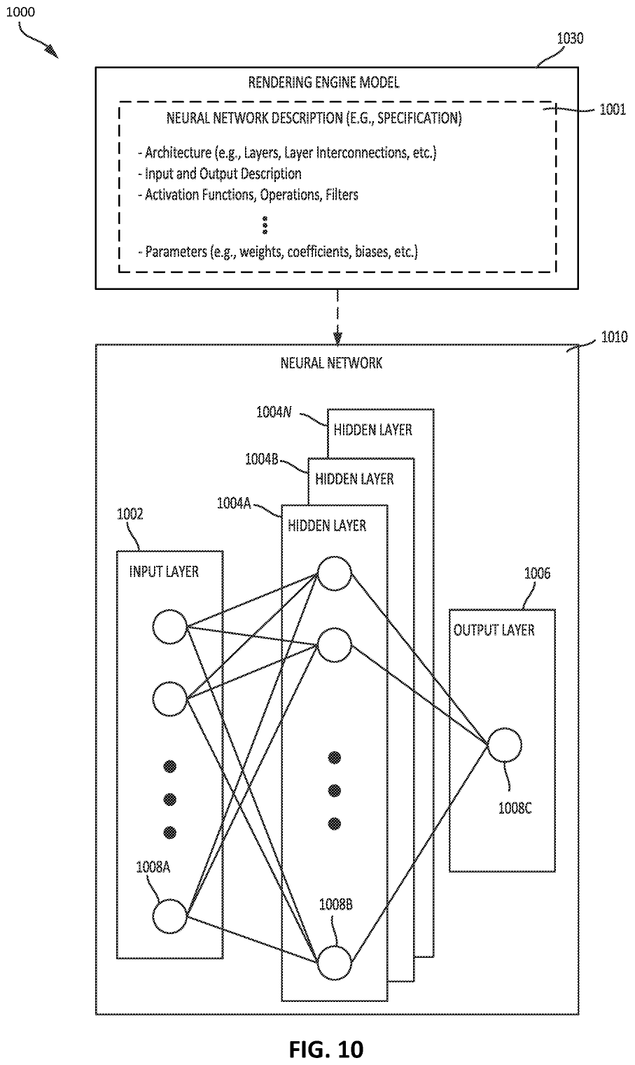

FIG. 10 illustrates an example neural network that may be utilized for segmentation of a 3-dimensional representation of a patient's face for removing an inner mouth region, according to some aspects of the present disclosure.

Architecture 1000 includes a neural network 1010 defined by an example neural network description 1001 in rendering engine model (neural controller) 1030. Neural network 1010 can represent a neural network implementation of a rendering engine for rendering media data. Neural network description 1001 can include a full specification of neural network 1010. For example, neural network description 1001 can include a description or specification of neural network 1010 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

In this example, neural network 1010 can include an input layer 1002, which includes input data, such as an image of the patient 104's face or a 2-dimensional projection of the 3-dimensional representation 402 of patient 104's face, etc.

Neural network 1010 may include hidden layers 1004A through 1004N (collectively “1004” hereinafter). Hidden layers 1004 can include n number of hidden layers, where n is an integer greater than or equal to one. The number of hidden layers can include as many layers as needed for a desired processing outcome and/or rendering intent. Neural network 1010 further includes an output layer 1006 that provides an output (e.g., identification of inner mouth region within an image of the patient 104's face or a 2-dimensional projection of the 3-dimensional representation 402 of patient 104's face) resulting from the processing performed by hidden layers 1004.

Neural network 1010 in this example can be a multi-layer neural network of interconnected nodes. Each node can represent a piece of information. Information associated with the nodes may be shared among the different layers and each layer retains information as information is processed. In some cases, neural network 1010 can include a feed-forward neural network, in which case there are no feedback connections where outputs of the neural network are fed back into itself. In other cases, neural network 1010 can include a recurrent neural network, which can have loops that allow information to be carried across nodes while reading in input.

Information can be exchanged between nodes through node-to-node interconnections between the various layers. Nodes of input layer 1002 can activate a set of nodes in first hidden layer 1004A. For example, as shown, each of the input nodes of input layer 1002 is connected to each of the nodes of first hidden layer 1004A. Nodes of hidden layer 1004A can transform the information of each input node by applying activation functions to the information. The information derived from the transformation can then be passed to and can activate the nodes of the next hidden layer (e.g., 1004B), which can perform their own designated functions. Example functions include convolutional, up-sampling, data transformation, pooling, and/or any other suitable functions. The output of the hidden layer (e.g., 1004B) can then activate nodes of the next hidden layer (e.g., 10041V), and so on. The output of the last hidden layer can activate one or more nodes of the output layer 1006, at which point an output is provided. In some cases, while nodes (e.g., nodes 1008A, 1008B, 1008C) in neural network 1010 are shown as having multiple output lines, a node has a single output and all lines shown as being output from a node represent the same output value.

In some cases, each node or interconnection between nodes can have a weight that is a set of parameters derived from training neural network 1010. For example, an interconnection between nodes can represent a piece of information learned about the interconnected nodes. The interconnection can have a numeric weight that can be tuned (e.g., based on a training dataset), allowing neural network 1010 to be adaptive to inputs and able to learn as more data is processed.

Neural network 1010 can be pre-trained to process the features from the data in input layer 1002 using the different hidden layers 1004 in order to provide the output through output layer 1006. In an example in which neural network 1010 is used to identify inner mouth region within 3-dimensional representation 402, neural network 1010 can be trained using training data that includes various 2-dimensional images with annotated inner mouth regions identified.

In some cases, neural network 1010 can adjust weights of nodes using a training process called backpropagation. Backpropagation can include a forward pass, a loss function, a backward pass, and a weight update. The forward pass, loss function, backward pass, and parameter update is performed for one training iteration. The process can be repeated for a certain number of iterations for each set of training media data until the weights of the layers are accurately tuned.

For a first training iteration for neural network 1010, the output can include values that do not give preference to any particular class due to the weights being randomly selected at initialization. For example, if the output is a vector with probabilities that the object includes different product(s) and/or different users, the probability value for each of the different product and/or user may be equal or at least very similar (e.g., for ten possible products or users, each class may have a probability value of 0.1). With the initial weights, neural network 1010 is unable to determine low level features and thus cannot make an accurate determination of what the classification of the object might be. A loss function can be used to analyze errors in the output. Any suitable loss function definition can be used.

The loss (or error) can be high for the first training dataset (e.g., images) since the actual values will be different than the predicted output. The goal of training is to minimize the amount of loss so that the predicted output comports with a target or ideal output. Neural network 1010 can perform a backward pass by determining which inputs (weights) most contributed to the loss of the neural network 1010, and can adjust the weights so that the loss decreases and is eventually minimized.

A derivative of the loss with respect to the weights can be computed to determine the weights that contributed most to the loss of neural network 1010. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

Neural network 1010 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. In other examples, neural network 1010 can represent any other neural or deep learning network, such as an autoencoder, a deep belief nets (DBNs), a recurrent neural networks (RNNs), etc.

FIG. 11 shows an example computing system, according to some aspects of the present disclosure. Example computing system 1100 of FIG. 11 can be used to implement any component described above with reference to FIGS. 1-10 including mobile device 102, servers 106, etc. Computing system 1100 can include components in electrical communication with each other using a connection 1105. Connection 1105 can be a physical connection via a bus, or a direct connection into processor 1110, such as in a chipset architecture. Connection 1105 can also be a virtual connection, networked connection, or logical connection.

In some embodiments computing system 1100 is a distributed system in which the functions described in this disclosure can be distributed within a datacenter, multiple datacenters, a peer network, etc. In some embodiments, one or more of the described system components represents many such components each performing some or all of the function for which the component is described. In some embodiments, the components can be physical or virtual devices.

Example system 1100 includes at least one processing unit (CPU or processor) 1110 and connection 1105 that couples various system components including system memory 1115, such as read only memory (ROM) 1120 and random-access memory (RAM) 1125 to processor 1110. Computing system 1100 can include a cache of high-speed memory 1112 connected directly with, in close proximity to, or integrated as part of processor 1110.

Processor 1110 can include any general purpose processor and a hardware service or software service, such as services 1132, 1134, and 1136 stored in storage device 1130, configured to control processor 1110 as well as a special-purpose processor where software instructions are incorporated into the actual processor design. Processor 1110 may essentially be a completely self-contained computing system, containing multiple cores or processors, a bus, memory controller, cache, etc. A multi-core processor may be symmetric or asymmetric.

To enable user interaction, computing system 1100 includes an input device 1145, which can represent any number of input mechanisms, such as a microphone for speech, a touch-sensitive screen for gesture or graphical input, keyboard, mouse, motion input, speech, etc. Computing system 1100 can also include output device 1135, which can be one or more of a number of output mechanisms known to those of skill in the art. In some instances, multimodal systems can enable a user to provide multiple types of input/output to communicate with computing system 1100. Computing system 1100 can include communications interface 1140, which can generally govern and manage the user input and system output. There is no restriction on operating on any particular hardware arrangement and therefore the basic features here may easily be substituted for improved hardware or firmware arrangements as they are developed.

Storage device 1130 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read only memory (ROM), and/or some combination of these devices.

The storage device 1130 can include software services, servers, services, etc., that when the code that defines such software is executed by the processor 1110, it causes the system to perform a function. In some embodiments, a hardware service that performs a particular function can include the software component stored in a computer-readable medium in connection with the necessary hardware components, such as processor 1110, connection 1105, output device 1135, etc., to carry out the function.

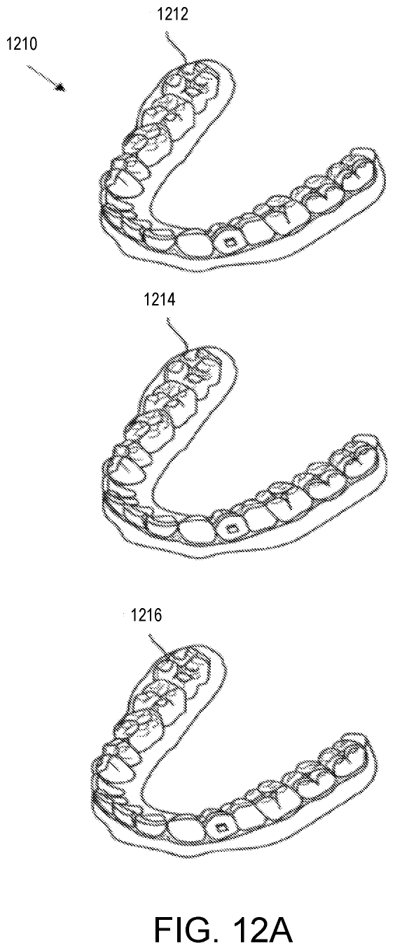

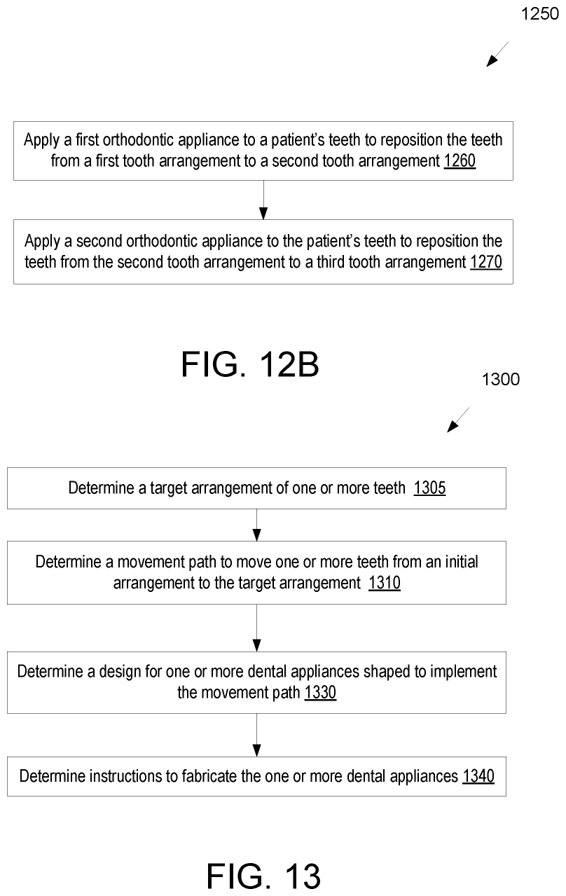

FIG. 12A illustrates a tooth repositioning system 1210 including a plurality of appliances 1212, 1214, 1216. The appliances 1212, 1214, 1216 can be designed based on generation of a sequence of 3D models of dental arches, which may be generated by performing intraoral scanning of a patient's oral cavity and performing registration and stitching between a plurality of intraoral scans generated from the intraoral scanning process. Any of the appliances described herein can be designed and/or provided as part of a set of a plurality of appliances used in a tooth repositioning system, and may be designed in accordance with an orthodontic treatment plan generated in accordance with embodiments of the present disclosure. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 1210 can include a first appliance 1212 corresponding to an initial tooth arrangement, one or more intermediate appliances 1214 corresponding to one or more intermediate arrangements, and a final appliance 1216 corresponding to a target arrangement. A target tooth arrangement can be a planned final tooth arrangement selected for the patient's teeth at the end of all planned orthodontic treatment, as optionally output using a trained machine learning model. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 1212, 1214, 1216 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

In an example of indirect fabrication, a mold of a patient's dental arch may be fabricated from a digital model of the dental arch generated by a trained machine learning model as described above, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 1212, 1214, 1216 after the digital models of the appliances 1212, 1214, 1216 have been processed by processing logic of a computing device. The processing logic may include hardware (e.g., circuitry, dedicated logic, programmable logic, microcode, etc.), software (e.g., instructions executed by a processing device), firmware, or a combination thereof.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a three dimensional (3D) virtual model of the patient's dental arch (mold). For example, a full scan of the mandibular and/or maxillary arches of a patient may be performed to generate 3D virtual models thereof. The intraoral scan may be performed by creating multiple overlapping intraoral images or scans from different scanning stations and then stitching together the intraoral images or scans to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner may determine a desired treatment outcome, which includes final positions and orientations for the patient's teeth. Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

In some instances, stereolithography (SLA), also known as optical fabrication solid imaging, is used to fabricate an SLA mold. In SLA, the mold is fabricated by successively printing thin layers of a photo-curable material (e.g., a polymeric resin) on top of one another. A platform rests in a bath of a liquid photopolymer or resin just below a surface of the bath. A light source (e.g., an ultraviolet laser) traces a pattern over the platform, curing the photopolymer where the light source is directed, to form a first layer of the mold. The platform is lowered incrementally, and the light source traces a new pattern over the platform to form another layer of the mold at each increment. This process repeats until the mold is completely fabricated. Once all of the layers of the mold are formed, the mold may be cleaned and cured.

Materials such as a polyester, a co-polyester, a polycarbonate, a thermopolymeric polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermopolymeric elastomer (TPE), a thermopolymeric vulcanizate (TPV) elastomer, a polyurethane elastomer, a block copolymer elastomer, a polyolefin blend elastomer, a thermopolymeric co-polyester elastomer, a thermopolymeric polyamide elastomer, or combinations thereof, may be used to directly form the mold. The materials used for fabrication of the mold can be provided in an uncured form (e.g., as a liquid, resin, powder, etc.) and can be cured (e.g., by photopolymerization, light curing, gas curing, laser curing, crosslinking, etc.). The properties of the material before curing may differ from the properties of the material after curing.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 1212, 1214, 1216 can be pressure formed or thermoformed over the molds. Each mold may be used to fabricate an appliance that will apply forces to the patient's teeth at a particular stage of the orthodontic treatment. The appliances 1212, 1214, 1216 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

Additional information may be added to the appliance. The additional information may be any information that pertains to the appliance. Examples of such additional information includes a part number identifier, patient name, a patient identifier, a case number, a sequence identifier (e.g., indicating which appliance a particular liner is in a treatment sequence), a date of manufacture, a clinician name, a logo and so forth. For example, after determining there is a probable point of damage in a digital design of an appliance, an indicator may be inserted into the digital design of the appliance. The indicator may represent a recommended place to begin removing the polymeric appliance to prevent the point of damage from manifesting during removal in some embodiments.

After an appliance is formed over a mold for a treatment stage, the appliance is removed from the mold (e.g., automated removal of the appliance from the mold), and the appliance is subsequently trimmed along a cutline (also referred to as a trim line). The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

FIG. 12B illustrates a method 1250 of orthodontic treatment using a plurality of appliances, in accordance with embodiments. The method 1250 can be practiced using any of the appliances or appliance sets described herein. In block 1260, a first orthodontic appliance is applied to a patient's teeth in order to reposition the teeth from a first tooth arrangement to a second tooth arrangement. In block 1270, a second orthodontic appliance is applied to the patient's teeth in order to reposition the teeth from the second tooth arrangement to a third tooth arrangement. The method 1250 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 13 illustrates a method 1300 for designing an orthodontic appliance to be produced by direct or indirect fabrication, in accordance with embodiments. The method 1300 can be applied to any embodiment of the orthodontic appliances described herein, and may be performed using one or more trained machine learning models in embodiments. Some or all of the blocks of the method 1300 can be performed by any suitable data processing system or device, e.g., one or more processors configured with suitable instructions.

At block 1305 a target arrangement of one or more teeth of a patient may be determined. The target arrangement of the teeth (e.g., a desired and intended end result of orthodontic treatment) can be received from a clinician in the form of a prescription, can be calculated from basic orthodontic principles, can be extrapolated computationally from a clinical prescription, and/or can be generated by a trained machine learning model such as treatment plan generator. With a specification of the desired final positions of the teeth and a digital representation of the teeth themselves, the final position and surface geometry of each tooth can be specified to form a complete model of the tooth arrangement at the desired end of treatment.

In block 1310, a movement path to move the one or more teeth from an initial arrangement to the target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set such as a 3D model of the patient's dental arch or arches can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, optionally including measured or extrapolated hidden surfaces and root structures, as well as surrounding bone and soft tissue.

Having both an initial position and a target position for each tooth, a movement path can be defined for the motion of each tooth. Determining the movement path for one or more teeth may include identifying a plurality of incremental arrangements of the one or more teeth to implement the movement path. In some embodiments, the movement path implements one or more force systems on the one or more teeth (e.g., as described below). In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In some embodiments, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be considered. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In block 1330, a design for one or more dental appliances shaped to implement the movement path is determined. In one embodiment, the one or more dental appliances are shaped to move the one or more teeth toward corresponding incremental arrangements. Determination of the one or more dental or orthodontic appliances, appliance geometry, material composition, and/or properties can be performed using a treatment or force application simulation environment. A simulation environment can include, e.g., computer modeling systems, biomechanical systems or apparatus, and the like. Optionally, digital models of the appliance and/or teeth can be produced, such as finite element models, 3D virtual models of the dental arches, etc. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

In block 1340, instructions for fabrication of the one or more dental appliances are determined or identified. In some embodiments, the instructions identify one or more geometries of the one or more dental appliances. In some embodiments, the instructions identify slices to make layers of the one or more dental appliances with a 3D printer. In some embodiments, the instructions identify one or more geometries of molds usable to indirectly fabricate the one or more dental appliances (e.g., by thermoforming plastic sheets over the 3D printed molds). The dental appliances may include one or more of aligners (e.g., orthodontic aligners), retainers, incremental palatal expanders, attachment templates, and so on.

The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by 3D printing a mold and thermoforming a plastic sheet over the mold.

Method 1300 may comprise additional blocks: 1) The upper arch and palate of the patient is scanned intraorally to generate three dimensional data of the palate and upper arch; 2) The three dimensional shape profile of the appliance is determined to provide a gap and teeth engagement structures as described herein.

Although the above blocks show a method 1300 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 1300 may be performed with any suitable fabrication system or device, such as the embodiments described herein. Some of the blocks may be optional, and the order of the blocks can be varied as desired.

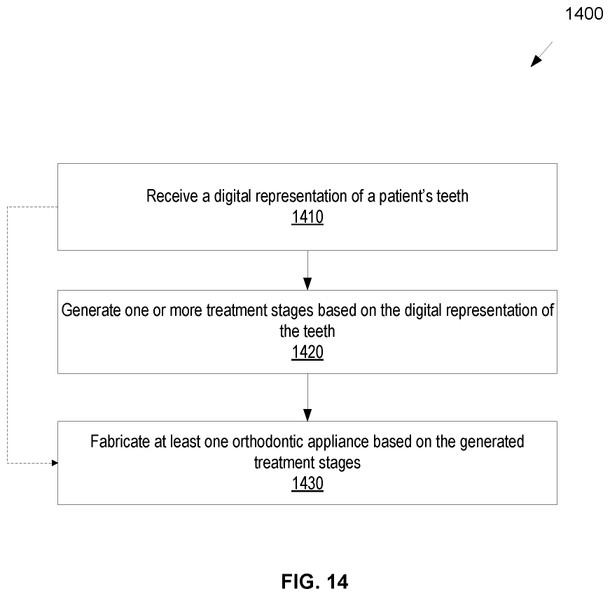

FIG. 14 illustrates a method 1400 for digitally planning an orthodontic treatment and/or design or fabrication of an appliance, in accordance with embodiments. The method 1400 can be applied to any of the treatment procedures described herein and can be performed by any suitable data processing system.

In block 1410, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 1420, one or more treatment stages are generated based on the digital representation of the teeth. Each treatment stage may include a generated 3D model of a dental arch at that treatment stage. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 1430, at least one orthodontic appliance is fabricated based on the generated treatment stages. For example, a set of appliances can be fabricated, each shaped according a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 14, design and/or fabrication of an orthodontic appliance, and perhaps a particular orthodontic treatment, may include use of a representation of the patient's teeth (e.g., receive a digital representation of the patient's teeth at block 1410), followed by design and/or fabrication of an orthodontic appliance based on a representation of the patient's teeth in the arrangement represented by the received representation.

For clarity of explanation, in some instances the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program, or a collection of programs that carry out a specific function. In some embodiments, a service can be considered a server. The memory can be a non-transitory computer-readable medium.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general-purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, solid state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

While the present disclosure is described with respect to the specific application of dental treatment planning for humans, the present disclosure is not limited thereto. The techniques described herein can equally be applied to any other medical application and planning. For example, techniques described can be utilized for treatment planning with respect to any other feature or element of human body such as eyes, nose, other facial elements, hands, legs, hips, foot, etc. Furthermore, the techniques described herein can equally be used for purposes of treatment planning for animals and their physical features just as in humans.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

What is claimed is:

1. A method comprising:
capturing media of a patient's face from multiple angles, using at least one device;
transforming the media into a 3-dimensional representation of the patient's face; and
transmitting the 3-dimensional representation of the patient's face to one or more processing components to be integrated with a 3-dimensional representation of the patient's intra-oral scan to visualize a dental treatment plan for the patient.

2. The method of claim 1, wherein the at least one device is a mobile device with a built-in camera.

3. The method of claim 2, wherein the media includes a plurality of 2-dimensional images of the patient's face captured by moving the mobile device around the patient's face.

4. The method of claim 1, wherein the media includes a video of the patient's face.

5. The method of claim 4, wherein the video is captured using a multi-camera system configured to capture images of the patient's face from a plurality of angles at the same time, the at least one device being the multi-camera system.

6. The method of claim 1, wherein the 3-dimensional representation of the patient's intra-oral scan is used as a reference for scaling the 3-dimensional representation of the patient's face.

7. The method of claim 1, wherein the media is captured via an application installed on the at least one device having access to a built-in camera of the at least one device.

8. The method of claim 7, wherein the application provides real-time guidance for moving the at least one device around the patient's face in order to optimize the media captured.

9. The method of claim 1, wherein the media includes a plurality of 2-dimensional images of the patient's face, and wherein transforming the media into the 3-dimensional representation of the patient's face comprises:
determining a plurality of camera poses, where each camera pose of the plurality of camera poses is determined for a 2-dimensional image of the plurality of 2-dimensional images;
generating a plurality of depth maps, where each depth map of the plurality of depth maps is generated for a 2-dimensional image of the plurality of 2-dimensional images based at least in part on the camera pose for the 2-dimensional image; and
generating the 3-dimensional representation of the patient's face based on combining information from the plurality of depth maps.

10. A method of generating a 3-dimensional representation of a patient's face for dental treatment planning, the method comprising:
receiving, at a processing component communicatively coupled to at least one device, a 3-dimensional representation of the patient's face based on media of the patient's face captured from multiple angles by the at least one device; and
integrating, at the processing component, the 3-dimensional representation of the patient's face with a 3-dimensional representation of the patient's intra-oral scan to visualize a dental treatment plan for the patient.

11. The method of claim 10, wherein integrating the 3-dimensional representation of the patient's face with the 3-dimensional representation of the patient's intra-oral scan comprises:
identifying an inner mouth region of the patient in the 3-dimensional representation of the patient's face;
removing the inner mouth region from the 3-dimensional representation of the patient's face;
determining a scaled-rigid relative transform for the 3-dimensional representation of the patient's face to the 3-dimensional representation of the patient's intra-oral scan;
replacing the inner mouth region removed from the 3-dimensional representation with the 3-dimensional representation of the patient's intra-oral scan; and
aligning the 3-dimensional representation of the patient's face with the 3-dimensional representation of the patient's intra-oral scan based on the scaled-rigid relative transform.

12. The method of claim 11, wherein the inner mouth region is identified in a 2-dimensional space using a machine learning-based model.

13. The method of claim 10, wherein the 3-dimensional representation of the patient's intra-oral scan provides a visualization of the patient's teeth after the dental treatment plan is completed.

14. A system comprising:
at least one device; and
a processing component communicatively coupled to the at least one device, wherein the at least one device is configured to:
capture media of a patient's face from multiple angles,
transform the media into a 3-dimensional representation of the patient's face, and
transmit the 3-dimensional representation of the patient's face to the processing component; and
the processing component is configured to:
integrate the 3-dimensional representation of the patient's face with a 3-dimensional representation of the patient's intra-oral scan to visualize a dental treatment plan for the patient.

15. The system of claim 14, wherein the processing component is further configured to receive the 3-dimensional representation of the patient's intra-oral scan from a dental scanner.

16. The system of claim 14, wherein the processing component is further configured to integrate with one or more dental treatment planning applications to use a visualization of the dental treatment plan.

17. The system of claim 14, wherein the at least one device is a mobile device with a built-in camera.

18. The system of claim 17, wherein the media includes a plurality of 2-dimensional images of the patient's faced captured by moving the mobile device around the patient's face.

19. The system of claim 14, wherein the media includes a video of the patient's face.

20. The system of claim 19, wherein the video is captured using a multi-camera system configured to capture images of the patient's face from a plurality of angles at the same time, the at least one device being the multi-camera system.

21. The system of claim 19, wherein a result of integrating the 3-dimensional representation of the patient's face with the 3-dimensional representation of the patient's intra-oral scan allows an operator of the at least one device to visualize movement of facial tissues, lips, and facial expressions once the dental treatment plan is applied to the patient's teeth.

22. The system of claim 14, wherein a mobile application on the at least one device is configured to provide real-time guidance for moving the at least one device around the patient's face in order to optimize the media captured.

23. The system of claim 14, wherein the processing component is configured to integrate the 3-dimensional representation of the patient's face with the 3-dimensional representation of the patient's intra-oral scan by:
identifying an inner mouth region of the patient in the 3-dimensional representation of the patient's face;
removing the inner mouth region from the 3-dimensional representation of the patient's face;
determining a scaled-rigid relative transform for the 3-dimensional representation of the patient's face to the 3-dimensional representation of the patient's intra-oral scan;
replacing the inner mouth region removed from the 3-dimensional representation with the 3-dimensional representation of the patient's intra-oral scan; and
aligning the 3-dimensional representation of the patient's face with the 3-dimensional representation of the patient's intra-oral scan based on the scaled-rigid relative transform.

24. The system of claim 23, wherein the inner mouth region is identified in a 2-dimensional space using a machine learning-based model.

25. The system of claim 14, wherein the processing component is configured to output a final result of integrating the 3-dimensional representation of the patient's face with the 3-dimensional representation of the patient's intra-oral scan to one or more dental planning tools for further analysis.

26. The system of claim 14, wherein the media includes a plurality of 2-dimensional images of the patient's face, and wherein to transform the media into the 3-dimensional representation of the patient's face the at least one device is to:
determine a plurality of camera poses, where each camera pose of the plurality of camera poses is determined for a 2-dimensional image of the plurality of 2-dimensional images;
generate a plurality of depth maps, where each depth map of the plurality of depth maps is generated for a 2-dimensional image of the plurality of 2-dimensional images based at least in part on the camera pose for the 2-dimensional image; and
generate the 3-dimensional representation of the patient's face based on combining information from the plurality of depth maps.
